(19) United States
(12) Patent Application Publication
(10) Pub. No.: US 2009/0069723 A1
(43) Pub. Date: Mar. 12, 2009

(54) Method for the analysis of oscillations generated in biological systems

(75) Inventor: Danielis Koganas, Schwerin (DE)

Correspondence Address: Vladimir Senatorov, 16206 E Phillips Drive, Englewood, CO, 80112, US

(73) Assignee: Alexandr Aronov, Saint-Petersburg (DE)

(21) Appl. No.: 11/658,640

(22) PCT Filed: Oct. 28, 2005
(86) PCT No.: PCT/DE2005/001951

§ 371 Date: Jan. 26, 2007

(30) Foreign Application Priority Data
Nov. 1, 2004 (DE) 10 2004 053 181.1

(51) Int. Cl. A61B 5/103

(52) U.S. Cl. 600/595

(57) ABSTRACT

Disclosed is a method for analyzing oscillations generated in biological systems, in which the change of conditions for the probability of a random event to occur compared in the previous cycle can be assessed by introducing an energetic component into the current cycle of the oscillation. In order to be able to do so, the analyzed signal is subdivided to the sequence of cycles in such a way that each subsequent cycle is provided with a common interval with the previous adjacent cycle. Such methods are used in the fields physiology, medicine, psychology and biocybernetics to analyze regulation in biological systems.

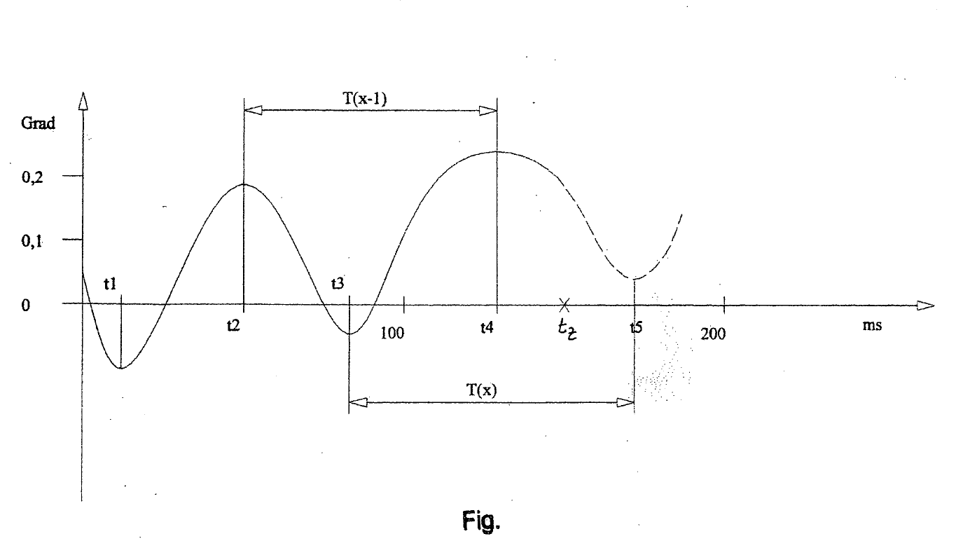

Invention belongs to the methods of research of control in biological systems applied in physiology, medicine, psychology and biocybernetics. The Germany Patent Office Publication De 10 2004 053 181 A1 of 11.05. 2006 refers it to the Cl.: A61B 5/04, A61B 5/16, A61B 5/0488. The international WIPO publication WO 2006/047996 A1 from the 11.05. 2006 refers it to the Cl.: GO6F 17/00 and A61B 5/11.

### BACKGROUND OF THE INVENTION

In physical biology the biological systems are sometimes considered as ensembles of interconnected and mutually entrained oscillators. According to A. S. Iberall's physical theory of homeokinesis, a stable organization is the consequence of the interaction of oscillatory processes at all levels of the system (A. S Iberall: Toward a general science of viable systems, McGraw-Hill, New York 1972).

Many registered signals that come from the brain, muscles or other parts of the body and can be received, for example, by electric, magnetic or mechanical sensors, are the consequences of such oscillatory activities. By means of the analysis of the oscillations resulting from the oscillatory activities it is possible to determine the features of physiological, mental, etc. processes and to detect the interactions of these processes among themselves. Thus the current system state can be estimated and corrected if necessary. The tremor research can be taken as an example of oscillations analysis for such purposes.

The tremor is a rhythmical, involuntary, oscillatory movement of a part of the body that is interpreted as a roughly sinusoidal movement. The tremor frequency lies between 8-12 Hz for normal people. The tremor is used as a symptom characterizing the neuromuscular system.

The initiations of discrete voluntary movements tend to start in the region around the physiological tremor phase which possesses a peak of speed in the direction of this voluntary movement. Voluntary movements of such kind could be facilitated by means of tremor (D. Goodman, J. A. Kelso: Exploring the functional significance of physiological tremor: A biospectroscopic approach, Exp. Brain Res. 49, S 419-431, 1983).

With Parkinsonian patients, the preferable phase of the initiation of discrete voluntary motor answers lies in the region of tremor which possesses the peak of speed in the direction opposite to this motor answer. If the voluntary initiation of the motor answer is executed as a reaction to the signal, then the reaction latent time depends on the initiation phase in the tremor cycle (G. Staude u.a.: Tremor as a factor in prolonged reaction times of Parkinsonian patients, Movement Disorders Vol. 10, Nr. 2, S 153-162, 1995).

In order to obtain the previously mentioned data on the interaction of the tremor with the motor output of system the researchers divided tremorograms into periods between the adjacent maxima. The initiation phase in the tremor cycle destroyed by the motor answer was calculated based on its length and the length of the previous tremor cycle. This approach did not take into account, that the probability of a signal appearance during the long tremor cycles was higher than during the short tremor cycles provided the signals appeared according to the random law. The experiment results confirm, that the average duration of the tremor cycle determined between the tremorograms local maxima is distinctly prolonged during the signal in comparison with the average duration of each one of the two previous tremor cycles determined in the same way (G. Staude; W. Wolf: Voluntary motor reactions: does stimulus appearance prolong the actual tremor period?, Journal of Electromyography and Kinesiology 9, S. 277-281, 1999). Now there is a question whether to interpret the increased statistical average value of the cycle duration as an inevitable error of the method of the average value determination or to investigate it as an attribute of the mechanism of control with the help of which the cognition of the stable organization of viable systems is possible.

The central origin of some frequencies participating in the formation of the tremor structure was proved quite recently (McAuley, I. C. Rotwell, C. D. Marsden: Frequency peaks of tremor, muscle vibration and electromyographic activity at 10 Hz, 20 Hz and 40 Hz during human finger muscle contraction may reflect rhythmicities of central neural firing, Exp. Brain 1997114: S. 525-541). This fact allows the assumption, that the changes of the correlations between the lengths of the two adjacent energy parts between the zero transitions of the first derivative, that can represent various cycles of tremors indicate the signs of a control existence, as well as the changes of correlations of cycles lengths.

Therefore in order to answer the above-mentioned question about the increased statistical average value of cycle duration it is necessary to take into account, that the signal appearance moment belongs to the period between the adjacent maxima and to the period between the adjacent minima at the same time. The lengths of these periods with a common part can be interpreted as a result of the mutual entrainments of the interconnected oscillators. Then the difference of durations of the mentioned periods characterizes the change of probability for the random event appearance in the second energy part of the subsequent period in comparison with the probability of its appearance in the second energy part of the previous period. And if the researchers believe, that the event can appear in the first energetic part of the cycle as well, as it is in the example of the analysis of the length of the cycle between the adjacent maxima, then it is necessary to take into consideration the possibility of a change of the length of this cycle because of the start of the reaction to the event. Hence, the indications of control of probability changes for the “capture” of the expected event by the oscillation structures should be sought in the difference of duration of the non-common parts smaller than the period. Similarly, the inversion time t for the “time invariance” test is chosen within the time range between the local maximum and the preceeding adjacent minimum in order to receive the greatest asymmetry value. A belonging of strategy of the waves formation processes to the certain tremors is distinguish reliably on the size of asymmetry (G. Deuschl u.a.: Tremor classification and tremor time series analysis, Chaos, Nr. 5 (1), S. 48-52, 1995).

The discussion on the approaches to the interpretation of experimental results leads to the conclusion, that in order to regard the existence of the above-mentioned probability changes as a manifestation of control it is necessary to investigate the behaviour of the duration of cycles constructed in another way or the behaviour of the cycle components measured between the local maximum and the previous local minimum and/or in the reverse order.

The registration of the hand tremor accelerations in the same direction is carried out by means of an ON/OFF sensor using instrumental measurement of the neuro-psycho-physical state of a person according to the EP 1095617. This corresponds to the determination of only one of the two components of a tremor cycle. However later these data are used for the estimation of tremor intensity during the studied time interval.

### BRIEF SUMMARY OF THE INVENTION

Therefore it is necessary to develop such a method of the analysis of oscillations generated in biological systems, that would contain an algorithm of the oscillations transformation and the obtained signals measurement, that would ensure the uniform conditions of the interpretation of the probability of the random signal appearance within the cycle and the uniform conditions of the interpretation of this signal influence on the oscillation formation. In order to solve this task, it is proposed to measure the time intervals between the adjacent transitions of the first derivative of the analyzed signal through zero; the resulting adjacent time intervals are then united into the sequences of cycles such, that each cycle consists of two above-mentioned adjacent time intervals, and the analyzed signal is divided into a sequence of cycles such, that each following cycle has a common time interval with the preceding adjacent cycle. This information can be obtained provided each time interval between a local maximum and the preceeding adjacent minimum (or the interval measured in the reverse order) is interpreted as constituting together one entity 2 components of two cycles. The new method of the analysis of oscillations generated in biological systems eliminates the above-mentioned shortcomings of the state-of-the-art. The method according to the current patent provides the advantage, that the various analyses of the change of duration of adjacent cycles or certain time intervals ensure the possibility to find out the strategies of control and their change depending on the changeable conditions.

### DETAILED DESCRIPTION OF THE INVENTION

The way of splitting of the analyzed signal into a sequence of cycles from which the parameters duration for the further studies is determined described in more detail in the execution example. For this purpose a separate plot shows a tremorogram pattern. In the tremorogram shown in the plot the analyzed signal before the appearance of a random signal at the moment of time tz is represented by a continuous line, and after its appearance by a dashed line. As can be seen from the plot, the tremor cycle Tx-1 is formed of the time interval between the local maximum t2 and the local minimum t3 and the time interval between the local minimum t3 and the local maximum t4. The order of time intervals between maxima and minima and intervals between minima and maxima in such tremor cycles changes as it can be seen e.g. in the series of tremor cycles Tx-2 (between the local minima t1 and t3), Tx-1, Tx.

The proposed method of the analysis of oscillations generated in biological systems can be widely applied in various areas of science, such as research of biological mechanisms of information processing, as well as for:
determination of common and individual ways of adaptation;
individual testing for determination of the actual abilities, e.g. of sportsmen;
state determination and training load control among highly skilled sportsmen;
evaluation of efficiency of the tuning on the performed action and correction of this tuning;
selection of a team (group) and/or parts of a team; research of animals reaction to the changeable environment as well as measurement and understanding of these reactions.

1. A method of the analysis of oscillations generated in biological systems in which the oscillations transformed into an amplified signal for further measurements are stored together with the irritants within the time of the recorded oscillations, in which the signals are filtered during the reproduction for the analysis, the intervals for the analysis are established, in the intervals local maxima and local minima are determined and numbered according to their order, the time intervals between each local minimum and the nearest adjacent maximum and the time intervals between each local maximum and the nearest adjacent minimum are measured and united into a sequences of cycles in such a way, that each cycle consists of two above-mentioned adjacent time intervals
characterized by that, that the analyzed signal is split into a sequence of cycles such, that each following cycle has a common time interval with a preceeding adjacent cycle.

2. A method according to claim 1,
characterized by that, that the differences between the adjacent time intervals of the adjacent cycles are used as analyzed parameters.

3. A method according to claim 2,
characterized by that, that the ratios of the adjacent time intervals of the adjacent cycles are used as analyzed parameters.

4. A method according to claim 3,
characterized by that, that the differences between the non-common time intervals of the adjacent cycles are used as analyzed parameters.

5. A method according to claim 4,
characterized by that, that the ratios of the non-common time intervals of the adjacent cycles are used as analyzed parameters.

6. A method according to claim 5,
characterized by that, that the ratios of the adjacent cycles durations are used as analyzed parameters.

7. A method according to claim 6,
characterized by that, that the differences between the adjacent cycles durations are used as analyzed parameters.
